(19) United States
(12) Patent Application Publication
(10) Pub. No.: US 2019/0290769 A1
(43) Pub. Date: Sep. 26, 2019

(54) Hydrogel Compositions

(71) Applicant: The Trustees of the University of Pennsylvania, Philadelphia, PA (US)

(72) Inventors: Kalil G. Abdullah, Philadelphia, PA (US); Leo L. Wang, Philadelphia, PA (US); Jason Alan Burdick, Philadelphia, PA (US)

(21) Appl. No.: 16/363,153

(22) Filed: Mar. 25, 2019

Related U.S. Application Data
(60) Provisional application No. 62/647,173, filed on Mar. 23, 2018.

(51) Int. Cl. A61K 47/34; A61P 35/00; A61K 9/19; A61K 31/19; A61K 9/06; A61K 47/40; A61K 9/00; A61K 31/7105

(52) U.S. Cl. CPC A61K 47/34; A61P 35/00; A61K 9/19; A61K 31/7105; A61K 9/06; A61K 47/40; A61K 9/0085; A61K 31/19

(57) ABSTRACT

Provided are compositions comprising a hydrogel formed from a cyclodextrin-modified branched polyethyleneimine and an adamantane-modified, eight-arm polyethyelene glycol. The hydrogel may contain an active agent, and can provide sustained release the active agent. Beneficial delivery characteristics may result from electrostatic or lipophilic interactions between the active agent and the hydrogel. Delivery of valproic acid from the present compositions can provide treatment following surgical resection of glioblastoma brain tumors.

### CROSS REFERENCE TO RELATED PATENT APPLICATION

The present application claims priority to U.S. Application No. 62/647,173, filed on Mar. 23, 2018, and incorporated herein by reference in its entirety for any and all purposes.

### GOVERNMENT RIGHTS

This invention was made with government support under Contract No. 1T32NS091006-01 awarded by the National Institutes of Health. The government has certain rights in the invention.

### TECHNICAL FIELD

The present disclosure pertains to compositions containing a hydrogel and an active agent.

### BACKGROUND

Glioblastoma is the most common primary malignant brain tumor and its prognosis remains poor. Historically, there have been several attempts at using local drug delivery to treat glioblastoma. These include convection enhanced delivery, direct tumor injection, and use of polymer wafers to deliver chemotherapeutics. Gliadel®, a polymeric chemotherapeutic wafer, is the only FDA-approved intracranial drug implant and treatment for recurrent and de novo glioblastoma, and showed initial promise in extending survival, but an unfavorable side effect profile has limited its widespread adoption.

Generally, the implantation of drug-eluting biomaterials within the brain is limited by several factors: implantable devices within the brain must be completely biocompatible, unable to migrate and cause mechanical damage to the ventricular system or local anatomical structures, and not “leak” the dissolving or migrating compound into the wound. Rigid larger scale polymers, such as polymeric wafers, do not meet these criteria and are prone to microshearing of surrounding tissue as they move within the brain cavity after implantation. These properties have resulted in clinical issues such as malignant brain edema and non-healing wounds. The need therefore exists for implantable materials that are capable of delivering an active ingredient to a sensitive anatomical location, such as the brain cavity, spinal column, or elsewhere, without damaging surrounding tissue.

### SUMMARY

Provided are compositions comprising a hydrogel formed from a cyclodextrin-modified branched polyethyleneimine and an adamantane-modified, eight-arm polyethylene glycol. Also disclosed are methods comprising administering to a subject such compositions.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

FIG. 1 illustrates how the instant hydrogels are formed from the self-assembly of two synthetic macromonomers.

FIG. 2 depicts the results of an assessment of glioma stem cell viability following exposure to a hydrogel according to the present invention that contains valproic acid, as compared with exposure to a hydrogel without that active agent.

FIG. 3 shows the results of the use of transwell assays to determine that cell viability following exposure to the present compositions containing valproic acid was significantly inhibited.

FIG. 4 provides the results of an assessment of cell toxicity of VPA alone, AD-PEG/CD-PEI alone, and the AD-PEG/CD-PEI hydrogel.

### DETAILED DESCRIPTION OF ILLUSTRATIVE EMBODIMENTS

The present invention may be understood more readily by reference to the following detailed description taken in connection with the accompanying figures and examples, which form a part this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “an active agent” is a reference to one or more of such inhibitors and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

As used herein, the terms “treatment” or “therapy” (as well as different word forms thereof) includes preventative (e.g., prophylactic), curative, or palliative treatment. Such preventative, curative, or palliative treatment may be full or partial. For example, complete elimination of unwanted symptoms, or partial elimination of one or more unwanted symptoms would represent “treatment” as contemplated herein.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired response in the individual, but also with factors such as the disease state or severity of the condition to be alleviated, hormone levels, age, sex, weight of the individual, the state of being of the patient, and the severity of the condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects. As an example, the compounds useful in the methods of the present invention are administered at a dosage and for a time such that the level of activation and adhesion activity of platelets is reduced as compared to the level of activity before the start of treatment.

Intracranial drug delivery for the treatment of brain tumors allows for higher concentrations of drugs to the brain than is possible by systemic therapy. The blood-brain barrier prevents molecular transport of drugs given systemically (orally or by intravenous therapy), and systemic delivery also results in significant toxicity. Attempts to deliver drugs into the brain directly has been limited by a lack of suitable drug selection and delivery vehicles. The present disclosure pertains to new, non-toxic hydrogel systems for sustained delivery of active agents, including for use in the treatment of malignant brain tumors.

There have been previous attempts at using hydrogel compounds for the treatment of solid tumors. The most recent of this is OncoGel™, which was an addition of paclitaxel, a chemotherapeutic, to a hydrogel (referred to as “ReGel”). This drug entered a Phase 2 clinical trial for the treatment of inoperable esophageal cancer (DuVall G A, Tarabar D, Seidel R H, Elstad N L, Fowers K D. Phase 2: a dose-escalation study of OncoGel (ReGel/paclitaxel), a controlled-release formulation of paclitaxel, as adjunctive local therapy to external-beam radiation in patients with inoperable esophageal cancer. Anticancer Drugs 2009; 20:89-95). This product did not complete clinical trials for FDA approval and based on interpretations of the data likely experienced the same problems as Gliadel®. More specifically, the use of a locally based chemotherapeutic compound tends to be more toxic than the use of a targeted biologic. Recent pre-clinical trials have also highlighted the role of hydrogels in the use of melanoma and transdermal injections (see Vishnubhakthula S, Elupula R, Duran-Lara E F. Recent Advances in Hydrogel-Based Drug Delivery for Melanoma Cancer Therapy: A Mini Review. J Drug Deliv 2017; 2017:7275985). Further reading reveals substantial recent interest in the role of hydrogels for the local treatment of tumors. Existing hydrogels lack sufficient stability, self-shearing, and self-assembling nature of our hydrogel, making them less attractive for direct intraoperative use (Norouzi M, Nazari B, Miller D W. Injectable hydrogel-based drug delivery systems for local cancer therapy. Drug Discov Today 2016; 21:1835-49).

The majority of hydrogel technology for cancer drug delivery remains pre-clinical. Further, hydrogel technology in neurosurgery is effectively in its infancy. The Wyss Institute at Harvard University is one industry leader in attempting to translate oncological therapy using hydrogels, but most of their technologies currently available for licensing remain regenerative in nature. The use of hydrogels more generally in medicine are limited to cosmetics and adjunctive agents for mechanical surgical use. These include CoSeal (Baxter) and Duraseal® (Integra LifeSciences Corporation) used for tissue bonding. The latter has found a home in neurosurgery and is used in many spine and cranial cases to seal the covering of the brain when primary closure is not complete. The market for these compounds is on the order of 100,000 to 200,000 cases yearly (low estimate). Though not a hydrogel and mostly fibrin based, the hemostatic agent FloSeal® shares many properties with hydrogel technology and has become nearly ubiquitous in operating rooms worldwide across all surgical specialties since 2000 (Baxter Scientific). The market for a specific glioblastoma targeted implant is 12,000 to 20,000 cases of primary and recurrent glioblastoma in the U.S. alone. A non-toxic and “low-risk” compound which can confer survival benefit would have a high likelihood of adoption, as previous trials and the currently available implant (Gliadel®) have a significant burden of side effects and toxicity.

To address the clinical need for an effective and safe intracranial implant to treat glioblastoma, the present inventors have developed a hydrogel-based implant composition that is capable of providing sustained-release of active agents. One agent of interest that can be delivered using the presently disclosed compositions is valproic acid, a histone deacetylase inhibitor (HDACi), for placement into the resection cavity after surgery. The in vitro studies described herein have shown that the novel valproic acid hydrogel is able to inhibit glioma stem and non-stem cell growth.

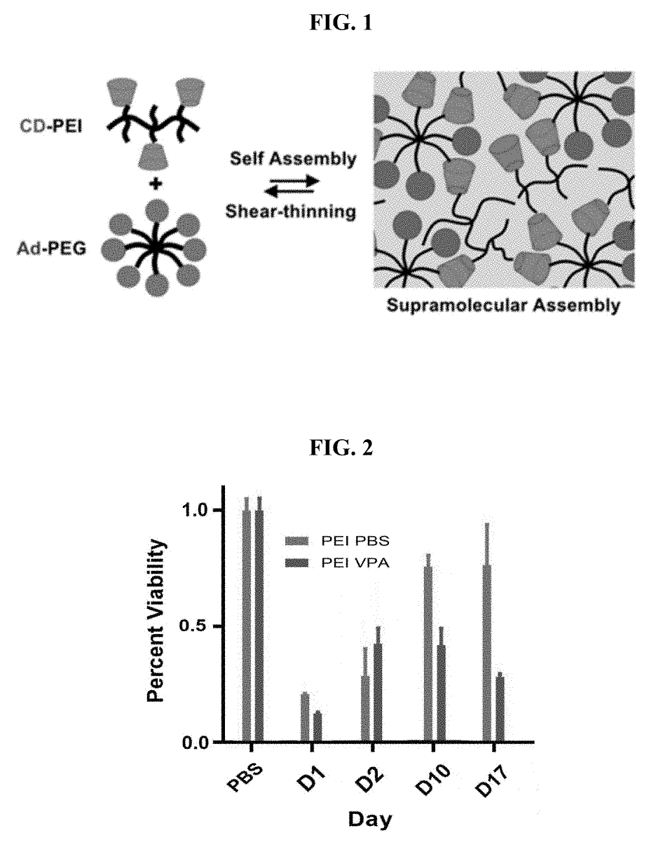

Accordingly, disclosed herein are compositions comprising a hydrogel formed from a cyclodextrin-modified branched polyethyleneimine and an adamantane-modified, eight-arm polyethylene glycol. Hydrogels are three-dimensional, cross-linked networks of water-soluble polymers. Macroscopically, they form a soft, semi-firm gel capable of adhering to a physical structure on contact (such as a tumor resection cavity), and they can deliver encapsulated therapeutics through combinations of hydrogel degradation and drug diffusion. The hydrogel of the present compositions is self-shearing and self-assembling, allowing delivery via microcatheter injection without subsequent modification or photoactivation. The hydrogel is formed from the self-assembly of two synthetic macromers in solution, cyclodextrin-modified branched polyethyleneimine (CD-PEI), and adamantane-modified 8-arm polyethylene glycol (Ad-PEG) (FIG. 1). These polymers have been previously described as nucleic acid carriers, but never in hydrogel form. It has also presently been found that, compared to PEI alone, the CD modification and the complexation with Ad-PEG significantly reduces cytotoxicity.

In the present hydrogels, CD and Ad interact through supramolecular hydrophobic complexation and confer shear-thinning due to their dynamic interaction that can be reversed under shear stress when extruded from a syringe. After extrusion and upon hydrogel deposition, these interactions allow the hydrogel to rapidly reassemble. The presently disclosed hydrogels were developed for delivery of active agents, wherein cationic (densely positively charged) PEI, a widely described transfection reagent, can be used to entrap and complex anionic (negatively charged) molecules (such as, for example, RNA molecules) for their local and sustained release, and promote their transfection into cells. The PEG component confers no charge.

It has been found that the Ad-PEG/CD PEI hydrogels of the present disclosure include a significant amount of positive charge due to the high PEI concentration (10 wt %), which confers an overwhelming amount of positive charge in the network. It was surprisingly discovered that this feature can be leveraged for sustained drug delivery. As described more fully herein, the instant compositions surprisingly conferred a sustained chemotherapeutic effect using valproic acid against glioma cells for almost three weeks. Similarly, RNAs, which are similarly anionic, were released from the present hydrogels and active for at least three weeks. There is also possibility of hydrophobic interactions between the lipophilic carbon chains of active agents (such as valproic acid) and cyclodextrin, which has been shown to interact with a wide range of small, lipophilic molecules. While not being bound to any particular theory of operation, this may also contribute to the sustained release effect. Thus, the present hydrogels confer a synergistic interaction with certain active agents that leads to sustained therapeutic effect due to electrostatic interaction and hydrophobic interaction.

Accordingly, the present compositions can contain an active agent. Any active agent can be used. In certain embodiments, the compositions contain an active agent that electrostatically or hydrophobically interacts with the hydrogel. In some instances, the active agent interacts both electrostatically and hydrophobically with the hydrogel. The active agent may be, for example, anionic at physiologic pH. This permits electrostatic interaction with the hydrogel, having the effects described above. As used herein, “physiologic pH” refers to the pH at the physiological location to which the composition containing the hydrogel is delivered when it is administered to a subject. Thus, when the composition is administered to a brain tumor cavity, “physiologic pH” will refer to the pH at the location of brain tumor cavity.

In certain embodiments, the hydrogel contains an active agent that is an RNA molecule. As noted above, RNA molecules bear an overall anionic charge. Thus, the hydrogel may contain an active agent that bears an anionic charge, including an overall anionic charge. In other instances, the hydrogel contains an active agent that contains a lipophilic functional group. An example of an active agent bearing a lipophilic functional group is valproic acid. Valproic acid is a small molecule that is anionic at physiologic pH due to its presence in its conjugate base form, valproate. Thus, there is electrostatic interaction between valproic acid and PEI, which is cationic.

Valproic acid may be present in the present compositions in a concentration of about 1 to about 10,000 μg/mL. For example, the valproic acid may be present in a concentration of about 10 to about 10,000, about 10 to about 7,500, about 10 to about 6,000, about 10 to about 5,000, about 10 to about 2,500, about 10 to about 2,000, about 10 to about 1,000, about 20 to about 1,000, about 25 to about 1,000, about 40 to about 1,000, about 50 to about 1,000, about 50 to about 750, about 50 to about 500, or about 100 to about 500 μg/mL, or may be present in a concentration of about 10, about 20, about 30, about 40, about 50, about 60, about 70, about 80, about 90, about 100, about 125, about 150, about 200, about 250, about 300, about 350, about 400, about 450, about 500, about 550, about 600, about 650, about 700, about 750, about 800, about 850, about 900, about 950, about 1000, about 1100, about 1250, about 1300, about 1400, about 1500, about 1750, about 2000, about 2500, about 3000, about 3500, about 4000, about 4500, about 5000, about 5500, about 6000, about 6500, about 7000, about 7500, about 8000, about 8500, about 9000, about 9500, or about 10,000 μg/mL.

As noted above, the instant composition can provide sustained release of an active agent. The sustained release can be for two or more days. For example, the composition can release the active agent over a period of about two days, about three days, about four days, about five days, about six days, about seven days (about one week), about eight days, about nine days, about ten days, about 11 days, about 12 days, about 13 days, about 14 days, about 15 days, about 16 days, about 17 days, about 18 days, about 19 days, about 20 days, about 21 days (about three weeks), about 22 days, about 24 days, about 26 days, about 28 days, about 29 days, or about 30 days (about one month).

Also provided herein are methods comprising administering a composition according to any of the embodiments described above. Administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In particular embodiments, the compositions are administered via injection. As noted above, the compositions are self-shearing and self-assembling, and therefore can be readily taken up within a syringe via shear thinning, delivered to an internal site of interested within a subject, and then will re-assemble into a cohesive hydrogel.

For oral therapeutic administration, the composition may be incorporated with a carrier, diluent, or excipient and used in the form of ingestible tablets, buccal tablets, troches, capsules, elixirs, suspensions, syrups, wafers, and the like. The amount of active agent(s) in such therapeutically useful compositions is preferably such that a suitable dosage will be obtained.

Liquid carriers, diluents, or excipients may be used in preparing solutions, suspensions, emulsions, syrups, elixirs, and the like. The compositions of this invention can be suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof.

Suitable examples of liquid carriers, diluents and excipients, for example, for oral, topical, or parenteral administration, include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof.

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. A dispersion can also be prepared in glycerol, liquid polyethylene glycols, and mixtures thereof and in oils. Under ordinary conditions of storage and use, these preparations may contain a preservative to prevent the growth of microorganisms.

Sterile injectable solutions may be prepared by incorporating the present composition in the pharmaceutically appropriate amounts, in any desired solvent, with various of the other ingredients enumerated above, as desired and required, followed by filtered sterilization.

Thus, the presently disclosed compositions may be administered in an effective amount by any of the conventional techniques well-established in the medical field. For example, the administration may be in the amount of about 0.1 mg/day to about 500 mg per day. In some embodiments, the administration may be in the amount of about 250 mg/kg/day. Thus, administration may be in the amount of about 0.1 mg/day, about 0.5 mg/day, about 1.0 mg/day, about 5 mg/day, about 10 mg/day, about 20 mg/day, about 50 mg/day, about 100 mg/day, about 200 mg/day, about 250 mg/day, about 300 mg/day, or about 500 mg/day.

The compositions may be administered to a subject to any desired internal location. In certain instances, the composition is administered to a tumor cavity in the subject following surgical tumor resection. For example, the composition may be administered to a brain tumor cavity in the subject following surgical tumor resection. The brain tumor that was removed by surgical tumor resection may be a glioblastoma tumor.

As described more fully in the examples below, the present methods can include subjecting the subject to radiation therapy following administration of the composition to the subject. The radiation therapy may be performed according to parameters (e.g., duration, type of radiation, location and scope of radiation) that are determined to be appropriate under the relevant circumstances. At least some of the radiation therapy should include exposure of the subject to radiation at the location to which the composition according to the present invention was administered.

Valprioic acid is presently indicated for use in treatment seizure disorders, mental/mood conditions (such as manic phase of bipolar disorder), and to prevent migraine headaches. The methods of the present disclosure may include administering the present compositions to a subject that suffers from migraine headaches, to a subject that has a seizure disorder, or to a subject with a bipolar disorder.

The following describes studies concerning the presently disclosed compositions that demonstrate that valproic acid-containing embodiments are able to inhibit glioma stem and non-stem cell growth, without being toxic to surrounding cells.

### Example 1—Method of Hydrogel Synthesis

Tosylated CD synthesis:

1. β-cyclodextrin (20 g, 17.62 mmol) was tosylated by reaction with p-toluenesulfonyl chloride (TosCl; 4.2 g, 22 mmol) in acetonitrile (10 mL) in de-ionized (DI) water for two hours.

2. Sodium hydroxide (2.18 g, 53.6 mmol) was dissolved in DI water and added dropwise and reaction stirred for 30 minutes.

3. pH was adjusted to 8.5 by addition of solid ammonium chloride.

4. The solution was cooled on ice and the precipitate collected.

5. The crude product was re-precipitated from cold water (3×200 mL), washed by acetone (3×50 mL) and dried under vacuum to afford the intermediate 6-o-monotosyl-6-deoxy-β-cyclodextrin as a white powder.

6. To form cyclodextrin-PEI, tosylated CD, 6-o-monotosyl-6-deoxy-β-cyclodextrin (736 mg, 0.572 mmol) was added dropwise to PEI (400 mg, 0.016 mmol) in 12 mL anhydrous DMSO via cannulation.

7. Triethylamine (121 mg, 166 uL, 1.2 mmol) was added with a syringe, the reaction was stirred for 72 hours at 70° C.

8. The reaction was dialyzed against DI H₂O for one week.

9. Product was frozen and lyophilized to afford CD-PEI. CD-PEI obtained under these conditions has a modification of 25 CD molecules per PEI and has a re-calculated molecular weight of ˜54,000 g/mol.

Ad-PEG synthesis:

1. 8-arm PEG-maleimide (20,000 g/mol, Creative PEGWorks) (100 mg, 5.0 μmol) was dissolved into 5 mL of DMSO.

2. ˜20 mg ((˜100 μmop of 1-adamantanethiol was dissolved into 20 mL of DMSO to react in excess.

3. PEG-maleimide in DMSO was added slowly to the 1-adamantanethiol reaction dropwise.

4. The reaction was stirred vigorously for hours at room temperature in a 50 mL round bottom flask.

5. At reaction completion, the product was dialyzed against DI water for one week.

6. By four days, unreacted adamantane precipitated out and was filtered from the product by vacuum filtration.

7. After one week of dialysis, the product was frozen and lyophilized to afford Ad-PEG. Ad-PEG has a modification of 8 Ad per PEG and has a molecular weight of 21,000 g/mol.

Hydrogel assembly:

1. Polymers were sterilized under UV irradiation for 1 hour prior to resuspension in PBS matching CD to Ad.

2. To form 100 μL gels at 20% wt, 8.9 mg of CD-PEI and 11.1 mg Ad-PEG were resuspended in PBS to match CD to Ad.

3. Polymers were briefly sonicated, vortexed and incubated at room temperature with valproic acid solutions for 30 minutes.

3. To form gels, polymer solutions were mixed manually, either alone or in valproic acid suspension.

### Example 2—In Vitro Testing

An injectable, guest-host assembled hydrogel between cyclodextrin-polyethyleneimine (CD-PEI) and adamantine-polyethylene glycol (Ad-PEG) was used as the base hydrogel as previously described (FIG. 1 & Example 1). To accommodate encapsulation of valproic acid (VPA) into the compound, CD-PEI and Ad-PEG ratios were titrated to maximize binding and release by altering weight by volume and formulation parameters. VPA introduction was iterative until drug solubility was maximized and stable at ranges of 15 to 40° C. to simulate operating room and intracavitary conditions. VPA concentration was finalized at 10 mg/ml in a 6% weight by volume compound. In other embodiments, VPA concentration was finalized at 20 mg/ml in a 20% weight compound.

In vitro testing of valproic acid on glioma stem cell and non-stem cells. The effects of valproic acid on T3565 cultured glioma stem cell viability was examined using MTS assays over varying concentrations and reliably occurred at an IC50 of 0.6 mg/ml. Dose-responses were also examined in U87 and U251 cells with similar results.

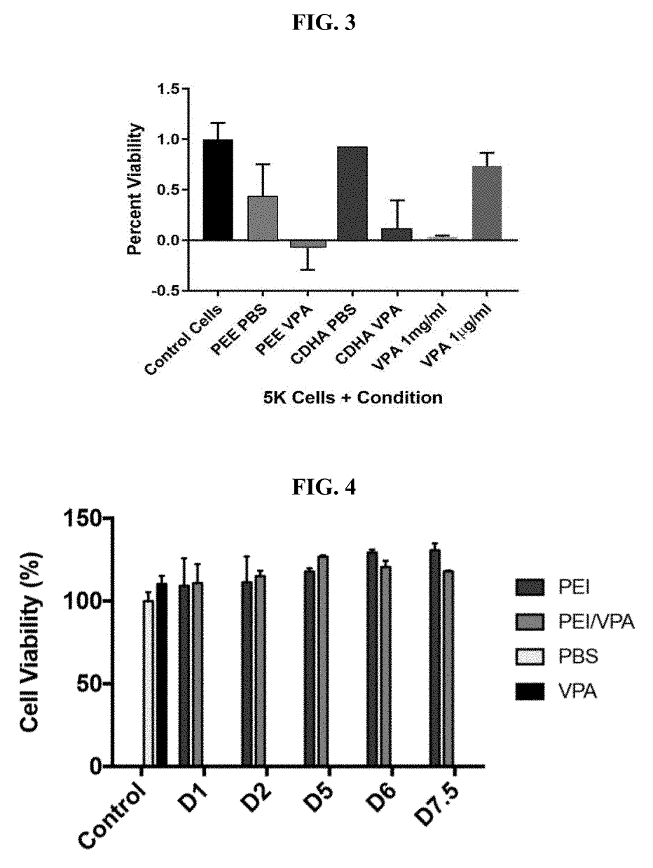

In vitro testing of hydrogel containing valproic acid. The delayed release of VPA from the shear thinning Ad-PEG/Cd-PEI hydrogel was evaluated via erosion chamber in multiple elutant assays run independently over three-week intervals (data not shown). VPA hydrogel of 40 μl was injected into the base of erosion chambers in triplicates and 1 ml PBS was added and harvested at pre-determined time intervals. The elutant was collected at these intervals and then added to pre-incubated T3565 glioma stem cells and evaluated by MTS assay. Cell viability was consistently less than 50% over a 17 day span on multiple testing (FIG. 2). The direct injection of hydrogels to cell culture to analyze 24-48 hour immediate effects on cell viability were also performed on T3565, U251, and U87 cells and consistently showed a significant (>80%) reduction in viability relative to control across all cell lines. Transwell migration assays were performed with T3565 cells and similarly showed significant reduction of cell viability (FIG. 3).

In an analysis of fibroblast cells, both valproic with and without the Ad-PEG/Cd-PEI delivery system were found to be non-toxic and did not affect cell viability via Alamar Blue fluorometry. NIH 3T3 fibroblasts were used as a surrogate for skin cells to determine cell toxicity. As visible per FIG. 4, there was no toxic effect of either VPA alone, AD-PEG/CD-PEI alone, or the AD-PEG/CD-PEI hydrogel.

### Example 3—Effect of Intracranial Injection of a VPA Hydrogel Compound on Tumor Progression in a Glioblastoma Mouse Model

Donor gliomas are formed by injection of DF-1 chicken fibroblasts transfected with RCAS-PDFG-B and RCAS-Cre plasmids and injected into Ntva; Ink4a-Arf−/−; PTEN(fl/fl); Gli-luc mice for induction of glioblastoma. Tumors are isolated, dissociated, and enzymatically digested to create single-cell suspensions. At 8 weeks, this cell suspension is stereotactically injected into the cortex of wild-type c-Met knockout mice. In non-control group mice, tumor cells are co-injected with 3 μl of: VPA-hydrogel, hydrogel carrier (no drug), or PBS. These groups are then segregated into subgroups undergoing no treatment, temozolomide injection only, radiation therapy only, and temozolomide+radiation therapy (n=10 for each group, n=120 mice in total, figures based on previous effect sizes). At two weeks after orthotopic injection, mice are injected with D-luciferin and imaged via bioluminescence to determine tumor size. Development of glioblastoma symptoms typically take 4-8 weeks after transplantation. Animals in appropriately assigned groups undergo radiation via the small animal research radiation platform and undergo intraperitoneal injection of temozolomide via established protocols.

What is claimed:

1. A composition comprising a hydrogel formed from a cyclodextrin-modified branched polyethyleneimine and an adamantane-modified, eight-arm polyethyelene glycol.

2. The composition according to claim 1, further comprising an active agent that electrostatically or hydrophobically interacts with said hydrogel.

3. The composition according to claim 1, further comprising an active agent that interacts electrostatically and hydrophobically with said hydrogel.

4. The composition according to claim 1, further comprising an active agent that is anionic at physiologic pH.

5. The composition according to claim 1, wherein said hydrogel contains an active agent that is an RNA molecule.

6. The composition according to claim 1, further comprising an active agent that contains a lipophilic functional group.

7. The composition according to claim 1, wherein said hydrogel contains an active agent that is valproic acid.

8. The composition according to claim 7, wherein said valproic agent is present in said composition in a concentration of about 1 to about 10,000 μg/mL.

9. The composition according to claim 8, wherein said valproic acid is present in said hydrogel in a concentration of about 50 to about 750 μg/mL.

10. The composition according to claim 8, wherein said valproic acid is present in said hydrogel in a concentration of about 50 to about 500 μg/mL.

11. The composition according to claim 1, wherein the composition is formulated for sustained release of an active agent.

12. The composition according to claim 11, wherein the composition releases the active agent over a period of at least one week.

13. The composition according to claim 11, wherein the composition releases the active agent over a period of at least two weeks.

14. The composition according to claim 11, wherein the composition releases the active agent over a period of up to one month.

15. A method comprising administering to a subject a composition according to claim 1.

16. The method according to claim 15, wherein the composition is administered via injection.

17. The method according to claim 15, wherein the composition is administered to a brain tumor cavity in said subject following surgical tumor resection.

18. The method according to claim 17, wherein the brain tumor was a glioblastoma tumor.

19. The method according to claim 17, further comprising subjecting the subject to radiation therapy.

20. The method according to claim 15, wherein the subject suffers from migraine headaches.

21. The method according to claim 15, wherein the subject has a seizure disorder.
